(19) United States
(12) Patent Application Publication
(10) Pub. No.: US 2016/0174939 A1
(43) Pub. Date: Jun. 23, 2016

(54) ULTRASONIC PROBE

(71) Applicant: SAMSUNG ELECTRONICS CO., LTD., Suwon-si (KR)

(72) Inventors: Kyung Il CHO, Seoul (KR); Jong Keun SONG, Yongin-si (KR)

(73) Assignee: SAMSUNG ELECTRONICS CO., LTD., Suwon-si (KR)

(21) Appl. No.: 14/879,188

(22) Filed: Oct. 9, 2015

(30) Foreign Application Priority Data
Dec. 19, 2014 (KR) 10-2014-0184621

(51) Int. Cl. A61B 8/00

(52) U.S. Cl. CPC A61B 8/4494; A61B 8/4444

(57) ABSTRACT

An ultrasonic probe includes: a transducer; a driving element electrically coupled to the transducer; a backing layer provided underneath the transducer and the driving element in a longitudinal direction of the ultrasonic probe, and configured to absorb heat generated from the transducer and the driving element and to absorb vibrations generated by the transducer; a heat spreader provided underneath the backing layer in the longitudinal direction of the ultrasonic probe and configured to absorb the heat from the backing layer; a heat pipe including a first contact portion contacting the heat spreader and a second contact portion in contact with the first contact portion; and a heat radiation plate configured to contact the second contact portion and transfer the heat from the heat spreader to an exterior of the ultrasonic probe.

### CROSS-REFERENCE TO RELATED APPLICATION

This application claims priority from Korean Patent Application No. 10-2014-0184621, filed on Dec. 19, 2014 in the Korean Intellectual Property Office, the disclosure of which is incorporated herein by reference in its entirety.

### BACKGROUND

1. Field

Apparatuses consistent with exemplary embodiments relate to an ultrasonic probe of an ultrasonic diagnostic apparatus.

2. Description of the Related Art

An ultrasonic diagnostic apparatus applies an ultrasonic signal from the surface of an object (for example, a human body) to a target inside of the body of the object, and non-invasively acquires tomograms of soft tissues or images regarding blood flow upon receiving reflected echo signals.

The ultrasonic diagnostic apparatus has compact size and low price, displays a diagnostic image in real time, as compared to other image diagnostic apparatuses, for example, an X-ray diagnostic apparatus, a computed tomography (CT) scanner, a magnetic resonance imaging (MRI) apparatus, and a nuclear medical diagnostic apparatus. In addition, because the ultrasonic diagnostic apparatus does not cause radiation exposure, the ultrasonic diagnostic apparatus may be safe. Accordingly, the ultrasonic diagnostic apparatus has been widely utilized for cardiac, abdominal, and urologic diagnosis as well as obstetric and gynecological diagnosis.

The ultrasonic diagnostic apparatus includes an ultrasonic probe for transmitting ultrasonic signals to a target object so as to acquire an ultrasonic image of the target of the object, and for receiving ultrasonic echo signals reflected from the target.

In the related art, as a transducer generating ultrasonic signals in the ultrasonic probe, a piezoelectric material, which converts electric energy into mechanical vibration energy to generate ultrasonic signals, is widely used.

On the other hand, in a transducer having a small number of channels, a heating value of about 1 W is generated by an electric circuit or the like to drive the probe, and such a heating value may be naturally emitted through a probe case. However, in a transducer having a large number of channels, an increased heating value of up to about 7 W is generated, and thus technologies to radiate and cool the ultrasonic probe are needed.

### SUMMARY

One or more exemplary embodiments provide an improved structure for effectively emitting heat generated by an ultrasonic transducer to the outside.

One or more exemplary embodiments also provide an improved structure for effectively absorbing ultrasonic waves emitted from an ultrasonic probe in a direction away from an object target.

Additional aspects of the disclosure will be set forth in part in the description which follows and, in part, will be obvious from the description, or may be learned by practice of the inventive concept.

In accordance with an aspect of an exemplary embodiment, there is provided an ultrasonic probe including: a transducer; a driving element electrically coupled to the transducer; a backing layer arranged in a downward direction of the transducer and the driving element in such a manner that the backing layer absorbs heat generated from the transducer and the driving element and also absorbs vibrations applied in a downward direction of the transducer and the driving element; a heat spreader provided below the backing layer so as to absorb heat applied to the backing layer; at least one heat pipe including a first contact portion contacting the heat spreader and a second contact portion for moving heat absorbed from the heat spreader to the outside; and at least one heat radiation plate configured to partially contact the second contact portion.

The second contact portion may include at least one bent portion.

The second contact portion may be extended in a longitudinal direction of the heat radiation plate after passing through peripheral parts of one end and the other end of a long side of the heat radiation plate.

The at least one heat radiation plate may include a first heat radiation plate and a second heat radiation plate arranged downwardly from the heat spreader.

The at least one heat pipe may include a plurality of second contact portions arranged to partially contact at least some parts of the first and second heat radiation plates so that heat is applied to the first and second heat radiation plates.

The plurality of second contact portions may include at least one bent portion.

The at least one heat pipe may further include: at least one connection portion bent and extended between the plurality of second contact portions in such a manner that the second contact portion contacting the first heat radiation plate is connected to the second contact portion contacting the second heat radiation plate.

The plurality of connection portions may be used, wherein the plural connection portions are arranged to correspond to the same side in a longitudinal direction of the heat radiation plate.

The plurality of heat pipes may be arranged to correspond to the first heat radiation plate and the second heat radiation plate.

The first contact portion may be extended in a longitudinal direction of the heat spreader and inserted into the heat spreader, and the heat pipe may further include an extension portion which is bent at the first contact portion and extended toward a heating portion.

The extension portion may be located in the heat spreader so that the heat pipe is bent in the spreader and passes through a bottom surface portion of the spreader.

The extension portion may be located at the outside of the heat spreader so that the heat pipe passes through one lateral surface portion of the spreader.

The plurality of extension portions may be used and located at both sides of the heat spreader, so that the heat pipe passes through both side portions of the spreader.

The heat spreader may include a contact portion contacting one surface of the backing layer, and the contact portion may include a micropattern having a plurality of holes.

The plural holes and the contact portion may be filled with a thermal grease or a phase change material.

The backing layer may have a thickness of 5 mm or less.

The heat spreader may further include: a seating portion in which a bottom surface portion and a lateral surface portion of the backing layer are seated in the heat spreader.

In accordance with an aspect of another exemplary embodiment, there is provided an ultrasonic probe includes: a housing configured to include a transducer, a body portion in which a driving element for driving the transducer is provided, and a handle portion extended from one side of the body portion; a backing layer arranged in a downward direction of the transducer and the driving element in such a manner that the backing layer absorbs heat generated from the transducer and the driving element and also absorbs vibrations applied in a downward direction of the transducer; a heat spreader provided below the backing layer so as to absorb heat applied to the backing layer; at least one heat radiation plate provided in the handle portion; and at least one heat pipe including a first contact portion inserted into the heat spreader and a second contact portion contacting the heat radiation plate.

The at least one heat radiation plate may be provided to correspond to a longitudinal direction of the handle portion.

The at least one heat radiation plate may include a first end adjacent to the body portion and a second end located at an opposite side of the first end in a longitudinal direction of the handle portion, and the second contact portion may be extended from the first end to the second end, passes through the second end, is bent at the second end, and is extended toward the first end.

The second contact portion may further include at least one bent portion bent between the first end and the second end.

The heat radiation plate may include a first heat radiation plate and a second heat radiation plate respectively corresponding to one side and the other side of the handle portion, and a plurality of second contact portions may respectively contact at least some parts of the first and second heat radiation plates.

The at least one heat pipe may further include: at least one connection portion bent and extended between the plurality of second contact portions in such a manner that the second contact portion contacting the first heat radiation plate is connected to the second contact portion contacting the second heat radiation plate.

The at least one heat pipe may include a plurality of heat pipes respectively corresponding to the first heat radiation plate and the second heat radiation plate.

The heat spreader may include a seating portion in which a bottom surface portion and a lateral surface portion of the backing layer are seated in the heat spreader, and a contact surface contacting the bottom surface portion of the backing layer of the seating portion may include a micropattern having a plurality of holes.

In accordance with an aspect of another exemplary embodiment, there is provided an ultrasonic probe including: a transducer; a driving element electrically coupled to the transducer; a backing layer contacting a bottom surface portion of the driving element in such a manner that the backing layer absorbs heat generated from the transducer and the driving element and also absorbs vibrations applied in a downward direction of the transducer and the driving element; a heat spreader having a contact portion that contacts a bottom surface portion of the backing layer so as to absorb heat applied to the backing layer; a heat pipe including one end inserted into the heat spreader; a heat radiation plate contacting at least some parts of the heat pipe; and a plurality of micro-sized holes arranged on a surface of the contact portion.

The plural holes and the contact portion may be filled with a thermal grease or a phase change material.

In accordance with an aspect of another exemplary embodiment, there is provided an ultrasonic probe including: a transducer; a driving element electrically coupled to the transducer; a backing layer provided underneath the transducer and the driving element in a longitudinal direction of the ultrasonic probe, and configured to absorb heat generated from the transducer and the driving element and to absorb vibrations generated by the transducer; a heat spreader provided underneath the backing layer in the longitudinal direction of the ultrasonic probe and configured to absorb the heat from the backing layer; a heat pipe including a first contact portion contacting the heat spreader and a second contact portion in contact with the first contact portion; and a heat radiation plate configured to contact the second contact portion and transfer the heat from the heat spreader to an exterior of the ultrasonic probe.

The second contact portion may include a bent portion.

The second contact portion may orthogonally extend from the first contact portion, in the longitudinal direction of the ultrasonic probe.

The heat radiation plate may include a first heat radiation plate and a second heat radiation plate arranged facing one another downwardly from the heat spreader along the longitudinal direction of the ultrasonic probe.

The second contact portion may be included into a plurality of second contact portions arranged to contact the first and second heat radiation plates and configured to transfer the heat to the first and second heat radiation plates.

The plurality of second contact portions may include a bent portion.

The heat pipe may further include: a connection portion which extends between the plurality of second contact portions so that one of the plurality of the second contact portions contacting the first heat radiation plate is connected to another one of the plurality of the second contact portions contacting the second heat radiation plate.

The connection portion may be included into a plurality of connection portions which are arranged in the longitudinal direction of the ultrasonic probe.

The heat pipe may be included into a plurality of heat pipes and the heat radiation plate is included into a plurality of heat radiation plates, and a number of the plurality of heat pipes may correspond to a number of the plurality of heat radiation plates.

The first contact portion may extend in a direction perpendicular to the longitudinal direction of the ultrasonic probe and is provided in the heat spreader, and the heat pipe may further include an extension portion which is bent at an end of the first contact portion and extends toward the heat radiation plate.

The extension portion may be provided in the heat spreader, and the heat pipe is bent in the heat spreader and passes through a bottom surface of the heat spreader.

The extension portion may be located at an exterior of the heat spreader, and the heat pipe may passes through a side surface of the heat spreader.

The extension portion may be included into a plurality of extension portions, and the plurality of extension portions may be located at opposite sides of the heat spreader and the heat pipe passes through opposite side portions of the heat spreader.

The heat spreader may include a contact portion contacting a bottom surface of the backing layer, and the contact portion may include a micropattern having a plurality of holes.

The plurality of holes and the contact portion may be filled with a thermal grease or a phase change material.

The backing layer may have a thickness of 5 mm or less.

The heat spreader may further include: a seating portion on which a bottom surface portion and a lateral surface portion of the backing layer are accommodated in the heat spreader.

In accordance with an aspect of another exemplary embodiment, there is provided an ultrasonic probe including: a housing including: a transducer, a body portion accommodating a driving element configured to drive the transducer, and a handle portion extending from the body portion; a backing layer underneath the transducer and the driving element in a longitudinal direction of the ultrasonic probe, the backing layer configured to absorb heat generated by the transducer and the driving element and configured to absorb vibrations generated by the transducer; a heat spreader provided underneath the backing layer in the longitudinal direction of the ultrasonic probe and configured to absorb heat absorbed by the backing layer from the transducer and the driving element; a heat radiation plate provided inside of the handle portion; and a heat pipe including: a first contact portion provided inside the heat spreader; and a second contact portion contacting the heat radiation plate.

In accordance with an aspect of another exemplary embodiment, there is provided an ultrasonic probe including: a transducer; a driving element electrically coupled to the transducer; a backing layer provided underneath the driving element in a longitudinal direction of the ultrasonic probe thereby contacting a bottom surface portion of the driving element, the backing layer configured to absorb heat generated from the transducer and the driving element and configured to absorb vibrations generated by the transducer; a heat spreader provided underneath the backing layer in the longitudinal direction of the ultrasonic probe, having a contact portion which contacts a bottom surface portion of the backing layer and configured to absorb heat absorbed by the backing layer; a heat pipe including a first end provided inside the heat spreader; a heat radiation plate contacting the heat pipe; and a plurality of micro-sized holes arranged on a surface of the contact portion, the surface of the contact portion contacting the bottom surface portion of the backing layer.

### BRIEF DESCRIPTION OF THE DRAWINGS

The above and/or other aspects of the disclosure will become apparent and more readily appreciated from the following description of the exemplary embodiments, taken in conjunction with the accompanying drawings of which:

FIG. 1 is a perspective view illustrating the external appearance of an ultrasonic probe according to an exemplary embodiment.

FIG. 2 is an exploded perspective view illustrating an ultrasonic probe according to an exemplary embodiment.

FIG. 3 is an enlarged perspective view illustrating components of an ultrasonic probe according to an exemplary embodiment.

FIG. 4 is a cross-sectional view illustrating components of the ultrasonic probe taken along line A-A′ of FIG. 1 according to an exemplary embodiment.

FIG. 5A is a cross-sectional view illustrating components of an ultrasonic probe according to an exemplary embodiment.

FIG. 5B is a cross-sectional view illustrating components of an ultrasonic probe according to an exemplary embodiment.

FIG. 6 is a perspective view illustrating an ultrasonic probe from which a housing of FIG. 1 is removed.

FIG. 7 is a side view illustrating components of an ultrasonic probe from which a heat radiation plate of FIG. 3 is removed.

FIG. 8A is a side view illustrating components of an ultrasonic probe from which a heat radiation plate of FIG. 3 is removed according to an exemplary embodiment.

FIG. 8B is a side view illustrating components of an ultrasonic probe from which a heat radiation plate of FIG. 3 is removed according to an exemplary embodiment.

FIG. 8C is a side view illustrating some constituent elements of an ultrasonic probe from which a heat radiation plate of FIG. 3 is removed according to an exemplary embodiment.

FIG. 9 is a conceptual diagram illustrating the operation principle of heat pipes shown in FIG. 2.

### DETAILED DESCRIPTION

Reference will now be made in detail to the exemplary embodiments, examples of which are illustrated in the accompanying drawings, wherein like reference numerals refer to like elements throughout.

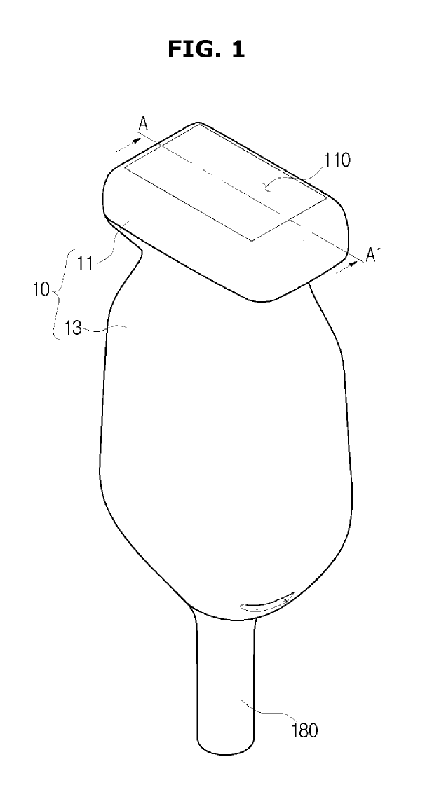

Referring to FIG. 1, on the basis of the shape of an ultrasonic probe 1 provided with a transducer 110 located on a top portion of the ultrasonic probe 1, a direction along which the transducer 110 is located is defined as an upward direction (i.e., a top side), and a direction along which a cable connection portion 180 is located is defined as a downward direction (i.e., a bottom side). On the basis of the line A-A′, a direction of a front portion is defined as a forward direction (i.e., a front side), and a direction of a rear portion is defined as a backward direction (i.e., a rear side).

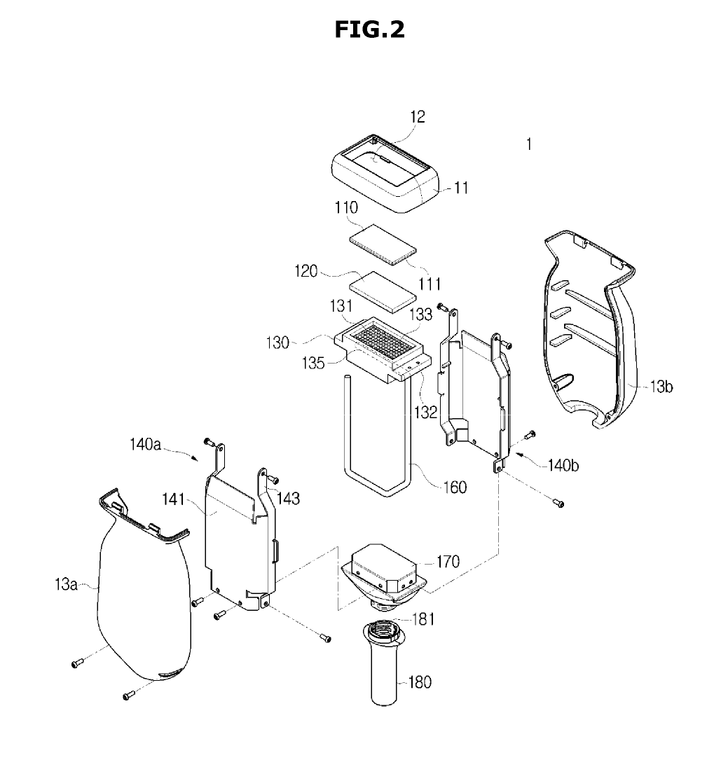

FIG. 1 is a perspective view illustrating the external appearance of an ultrasonic probe 1 according to an exemplary embodiment. FIG. 2 is an exploded perspective view illustrating an ultrasonic probe 1 according to an exemplary embodiment.

Referring to FIGS. 1 and 2, an ultrasonic probe 1 includes a housing 10 forming the external appearance thereof, a transducer 110 generating ultrasonic signals from the inside of the housing 10, and a heat spreader 130 absorbing heat generated by the transducer 110.

The housing 10 may include a body portion 11 and a handle portion 13. The body portion 11 is combined with the handle portion 13, resulting in forming of the external appearance of the ultrasonic probe 1 and accommodating various components including a transducer 110, a heat spreader 130, other electronic components, etc. In addition, the body portion 11 and the handle portion 13 are combined with each other, resulting in formation of an internal space in which the various components are accommodated.

An opening 12 may be formed in the body portion 11. The opening 12 may be provided at an upper portion end of the body portion 11 (i.e., a top end), and may be used as a passage through which ultrasonic signals generated by the transducer 110 propagates. The opening 12 may have a shape corresponding to the transducer 110.

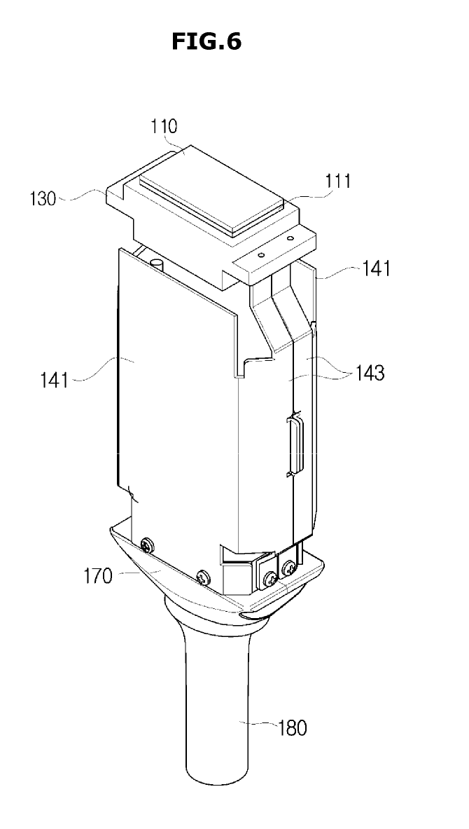

The handle portion 13 may include a front handle portion 13a and a rear handle portion 13b. The front handle portion 13a may be symmetrical to the rear handle portion 13b. The front handle portion 13a may be combined with the rear handle portion 13b, resulting in formation of an internal space in which one or more heat radiation plates 140 (see FIG. 6) including a first heat radiation plate 140a and a second heat radiation plate 140b is located.

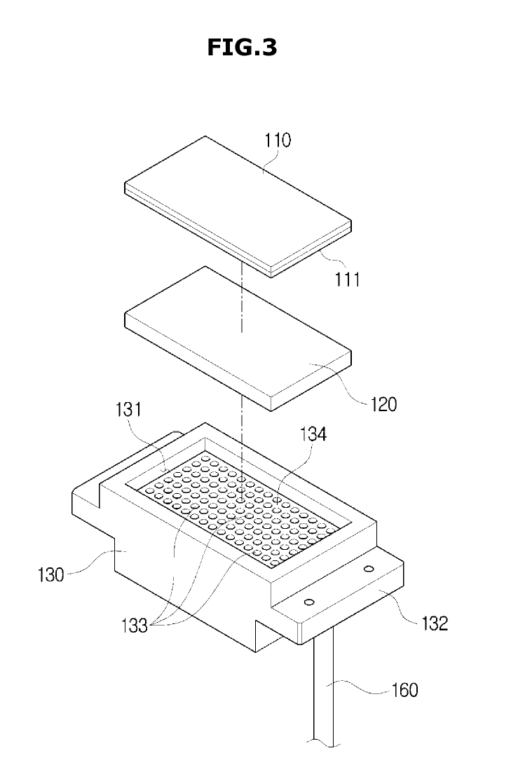

Referring to FIGS. 2 to 4, side surfaces of the transducer 110 and the opening 12 may be provided to face each other from the front-to-rear viewpoint of the housing 10. In an exemplary embodiment, the transducer 110 may be a magnetostrictive ultrasonic transducer using a magnetostrictive effect of a magnetic substance which is mainly used in an ultrasonic probe 1, a piezoelectric ultrasonic transducer (PZT transducer) using a piezoelectric effect of a piezoelectric substance such as lead zirconate titanate (hereinafter referred to as PZT), or the like may be used as the transducer 110. In addition, a capacitive micromachined ultrasonic transducer (hereinafter referred to as “cMUT”) which transmits and receives ultrasonic signals using vibrations of several hundred or thousands of micromachined thin films may also be used as the transducer 110. The following description assumes that the transducer 110 corresponds to a piezoelectric ultrasonic transducer including a PZT. Specifically, a 2D ultrasonic transducer based on a PZT will hereinafter be described in detail. However, it should be noted that the exemplary embodiment of the transducer 110 applied to the ultrasonic probe 1 of may not be limited to the piezoelectric ultrasonic transducer.

A driving element 111 having a direct circuit for driving the transducer 110 is bonded to the transducer 110, and may be provided at a bottom surface of the transducer 110 (i.e., an inner side of the transducer 110). In accordance with an exemplary embodiment, the direct circuit may be implemented as an Application Specific Integrated Circuit (ASIC) driving circuit 111. The ASIC driving circuit is electrically coupled to the transducer 110 so that the transducer 110 is driven and various electrical signals can be controlled.

A backing layer 120 may be provided at a bottom surface (i.e., an inner side) of the driving element 111. The backing layer 120 absorbs vibrations transferred from the transducer 110 in a downward direction (i.e., toward an inner side of the ultrasonic probe 1), and suppresses redundant vibration. The backing layer 120 is formed of a material composed of large-diameter particles such as rubber, so that the backing layer 120 can effectively absorb vibrations.

The heat spreader 130 may be located at the bottom of the backing layer 120 (i.e., an inner side). The heat spreader 130 may be formed to absorb heat generated from the transducer 110 and the driving element 111 to the backing layer 120.

As described above, the backing layer 120 includes a material formed of large-sized particles, so that it has low thermal conductivity and much heat not applied to the outside. As a result, the backing layer 120 is unfavorable in cooling the transducer 110. In order to quickly conduct heat from the backing layer 120 and transmit the conducted heat to the exterior of the probe 1, the heat spreader 130 may include a metal such as aluminum having superior thermal conductivity than the material contained in the backing layer 120. The heat spreader 130 may include a seating portion 131 in which the backing layer 120 is seated. The seating portion 131 may be formed in a hexahedral groove corresponding to a bottom part and a side part of the backing layer 120. The hexahedral groove is recessed toward the inside of the heat spreader 130.

When the backing layer 120 is seated in the heat spreader 130, the seating portion 131 may be provided to contact the bottom part and the side part of the backing layer 120. Accordingly, heat is transferred from the backing layer 120 to the heat spreader 130 through thermal conduction.

Specifically, the seating portion 131 includes a contact portion 134 facing the bottom surface of the backing layer 120 which has the largest area of the backing layer 120, and micropatterns including a plurality of micro-sized holes 133 may be formed in the contact portion 134.

As described above, the backing layer 120 must maintain a predetermined depth (or thickness) along a direction extending between the transducer 110 and the cable connection portion 180, so that the backing layer may absorb vibrations generated in a direction perpendicular to the thickness direction (i.e., the direction extending between the transducer 110 and the cable connection portion 180). However, the backing layer 120 has low thermal conductivity. The depth or thickness of the backing layer 120 is proportional to the amount of heat capable of being stored in the backing layer 120. As a result, the backing layer 120 is unfavorable in cooling the overall ultrasonic probe 1.

Therefore, in order to increase the cooling capability of the ultrasonic probe 1, the thickness of the backing layer 120 is reduced. In order to maintain the absorption capability of vibrations, micropatterns may be located in the contact portion 134 contacting the bottom surface of the backing layer 120.

Because the backing layer 120 has a small thickness, vibrations, which are not absorbed in the backing layer 120 and penetrate the backing layer 120, arrive at a plurality of micro-sized holes 133. Vibrations enter the inside of several holes 133 and are scattered, so that residual vibrations can be suppressed. The depth of the backing layer 120 may be 5 mm or less, preferably, 2 mm-3 mm.

Thermal grease or a phase change material, such as a thermal medium having superior thermal conductivity, may be applied to the contact portion 134 and the internal space of several holes 133.

According to an exemplary embodiment, a plurality of holes 133 may be formed in a cylindrical shape. However, the exemplary embodiment is not limited thereto, and each hole may also be formed in a semicircle or square pillar shape. In addition, for convenience of description and better understanding of the inventive concept, FIGS. 2 to 5B illustrate the enlarged view of the plurality of holes 133.

A coupling portion 132 may be provided at one side of the heat spreader 130. The coupling portion 132 may protrude from opposite sides of the heat spreader 130. The coupling portion 132 may be coupled to an inner lateral surface of the body portion 11 of the housing 10. The coupling portion 132 is coupled to the inner lateral surface of the body portion 11, so that the heat spreader 130 may be coupled to and supported by the body portion 11.

For example, the backing layer 120 may be inserted into a space formed between the seating portion 131 and the body portion 11. As a result, the backing layer 120 may be fixed to a predetermined position without being coupled to the body portion 11 and the heat spreader 130.

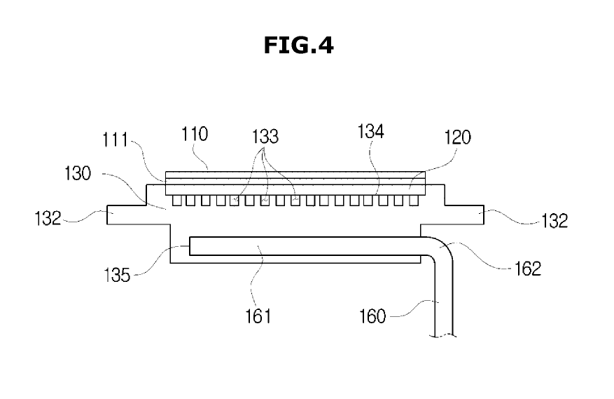

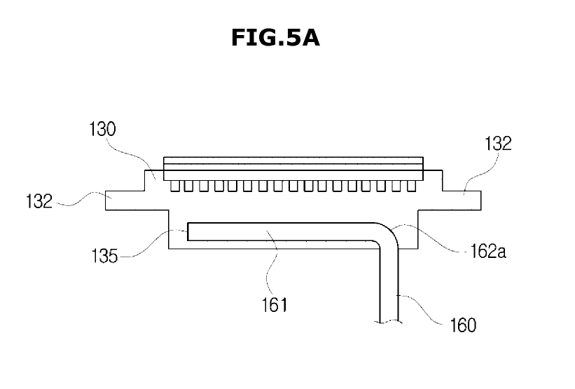

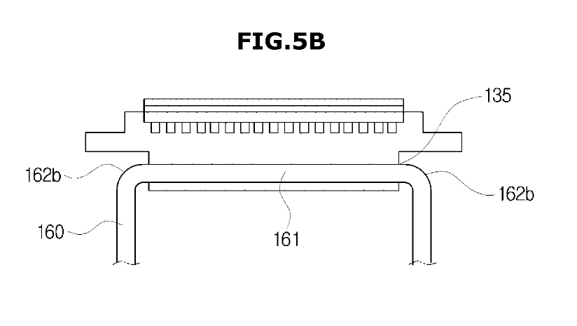

The heat spreader 130 may further include an insertion groove 135 (see FIGS. 4 and 5). The insertion groove 135 may provide a space in which a heat pipe 160 may be inserted. The insertion groove 135 is provided at the heat spreader 130 in such a manner that heat can be efficiently transferred from the heat spreader 130 to the heat pipe 160, and the depth of the insertion groove 135 may reach a surface on the condition that the heat spreader 130 thermally contacts the backing layer 120.

Referring to FIGS. 2 and 6, the ultrasonic probe 1 may further include a heat radiation plate 140. The heat radiation plate 140 may be coupled to the heat spreader 130 through the heat pipe 160. The heat radiation plate 140 may be used as a passage through which heat generated from the heat spreader 130 is transferred to the exterior of the ultrasonic probe 1.

The heat radiation plate 140 may include a first heat radiation plate 140a and a second heat radiation plate 140b. The first heat radiation plate 140a and the second heat radiation plate 140b may be respectively located at the front part and the rear part of the inside of the handle portion 13.

The heat radiation plate 140 may include a heat radiation plate body 141 and a heat radiation plate coupling portion 143.

The heat radiation plate body 141 is spaced apart from the inside of the handle portion 13 by a predetermined distance. A front surface of the heat radiation plate body 141 and one side of the front surface of the heat radiation plate body 141 may be curved according to the external appearance of the housing 1, differently from the above exemplary embodiment. One side of a lower part of the heat radiation plate body 141 may be coupled to a lower part 170 of the probe.

The heat radiation plate coupling unit 143 may be coupled to receive heat from the heat spreader 130, separately from heat received from the heat pipe 160. The heat radiation plate coupling unit 143 may be extended upwardly (i.e., toward the top side of the ultrasonic probe 1) from a side surface of the heat radiation plate body 141. The heat radiation plate coupling unit 143 may be extended upwardly from both sides of the heat radiation plate body 141, and may be coupled to the heat spreader 130.

For example, an upper end of the heat radiation plate coupling unit 143 may be rounded. As a result, even when the position of the heat radiation plate 140 moves to another position, the heat radiation plate 140 is coupled to the heat radiation plate coupling unit 143 and may rotate within a predetermined range.

Referring to FIGS. 4 to 8C, the ultrasonic probe 1 may further include a heat pipe 160. One end portion (i.e., a first end portion) of the heat pipe 160 may be coupled to the heat spreader 130, and the other end portion (i.e., a second end portion) thereof may contact the heat radiation plate 140. In more detail, the heat pipe 160 may include a first contact portion 161 inserted into the insertion groove 135 of the heat spreader 130, an extension portion 162 extending from the first contact portion 161 and bent toward the heat radiation plate 140, and a second contact portion 163 contacting the heat radiation plate body 141 so as to transfer heat via thermal conduction. The heat pipe 160 is thermally coupled between the heat spreader 130 and the heat radiation plate 140, and heat of the heat spreader 130 transfers to the heat radiation plate 140, so that heat can be radiated toward the exterior of the ultrasonic probe 1.

The first contact portion 161 may be formed to absorb and transfer heat from the heat spreader 130 to the heat pipe 160. Accordingly, the first contact portion 161 is extended in a longitudinal direction of the heat spreader 130 (which is perpendicular from a longitudinal direction of the ultrasonic probe), and the extension range may have a length of a bottom side surface of the backing layer 120 thermally contacting the backing layer 120. However, the insertion direction of the first contact portion 161 is not limited thereto, and may also be perpendicular (i.e., the longitudinal direction of the ultrasonic probe) to the longitudinal direction of the heat spreader 130.

Referring to FIG. 4, the extension portion 162 is extended from the first contact portion 161, and is bent to extend in the longitudinal direction of the ultrasonic probe 1. The heat pipe 160 bent by the extension portion 162 may be coupled to the second contact portion 163.

The extension portion 162 may be provided at one side of the heat spreader 130. Therefore, the heat pipe 160 may be bent toward the second contact portion 163 which passes through one lateral surface of the heat spreader 130 and contacts the heat radiation plate 140.

FIGS. 5A and 5B illustrate different exemplary embodiments of the extension portion 162. The extension portion 162, the first contact portion 161 contacting the extension portion 162, the heat spreader 130, and other constituent elements are identical to those of the above-mentioned exemplary embodiment, and additional description of the other constituent elements will herein be omitted for convenience of description.

Referring to FIG. 5A, the extension portion 162a may be provided in the heat spreader 130. Therefore, the heat pipe 160 may be bent toward the second contact portion 163 that passes through a bottom side surface of the heat spreader 130 and contacts the heat radiation plate 140.

Referring to FIG. 5B, a plurality of extension portions 162b may be provided. The extension portions 162b may be respectively provided near both lateral surfaces of the heat spreader 130. Therefore, the heat pipe 160 may pass through both lateral surfaces of the heat spreader 130, be bent toward the second contact portion 163, and be connected to the second contact portion 163. In this case, both ends of the heat pipe 160 may be provided as the second contact portion 163.

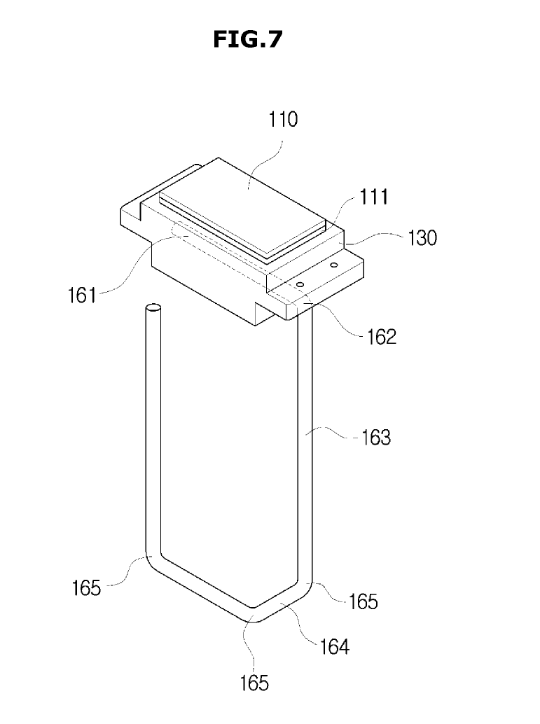

Referring to FIGS. 6 and 7, the second contact portion 163 may be extended to contact the inner lateral surface of the heat radiation plate body 141. The second contact portion 163 may be a section through which heat is transferred to the heat radiation plate 140 through thermal conduction.

The second contact portion 163 may include at least one bent portion 165 bent and extended so that a region having a large amount of surface area of the second contact portion 163 contacts the heat radiation plate body 141. That is, the second contact portion 163 may be extended in a longitudinal direction of the heat radiation plate 140, may be orthogonally bent by the bent portion 165 with respect to the longitudinal direction of the heat radiation plate 140, so that the second contact portion 163 may be arranged in a vertical direction (i.e., a longitudinal direction of the ultrasonic probe) with respect to the longitudinal direction of the heat radiation plate 140. The second contact portion 163 extended in a vertical direction may be orthogonally bent by another bent portion 165 with respect to the vertical direction of the longitudinal direction, so that the second contact portion 163 may be extended in the longitudinal direction of the heat radiation plate 140.

If a plurality of heat radiation plates 140 is provided, a plurality of second contact portions 163 may be provided. For this purpose, each of the second contact portions 163 may include a connection portion 164 having a bent portion 165 capable of being coupled to each second contact portion 163.

Although the connection portion 164 does not directly contact the heat radiation plate 140, the heat pipe 160 contacts a plurality of heat radiation plates 140 so that heat can be transferred through the heat pipe 160.

That is, heat received from the heat spreader 130 by the first contact portion 161 is applied to the second contact portion 163 through the extension portion 162. Primarily, the second heat radiation plate 140b contacts one of the second contact portions 163, so that heat can be applied to the second heat radiation plate 140b. Heat isothermally moves along the heat pipe 160, passes through a section corresponding to the second heat radiation plate 140b, and moves to the other one of the second contact portions 163 contacting the first heat radiation plate 140a through the connection portion 164, so that heat can move to the first heat radiation plate 140a.

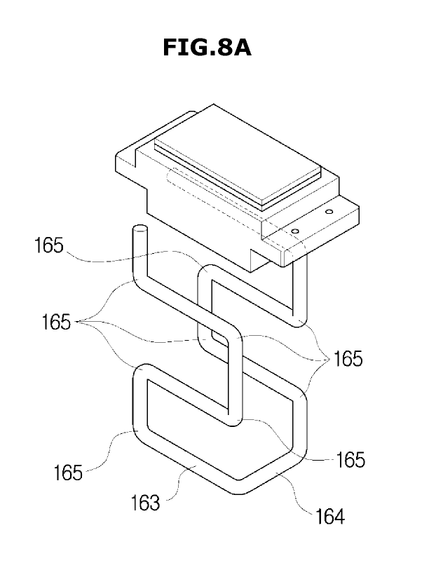

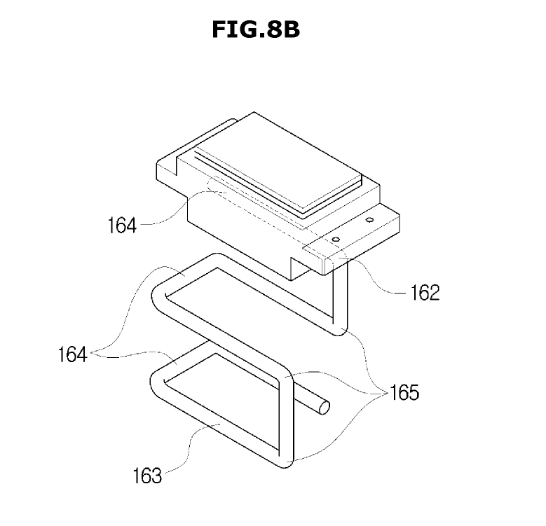

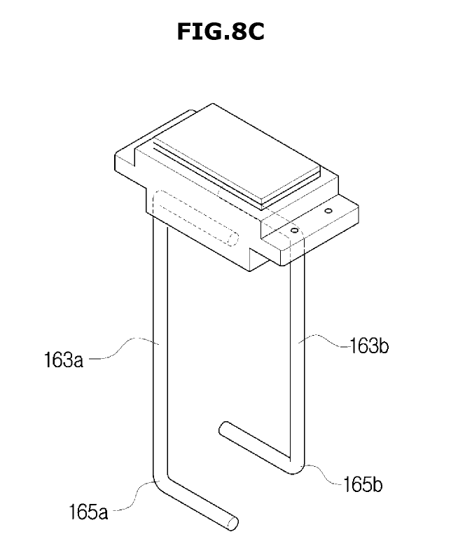

FIGS. 8A to 8C illustrate arrangement of the heat pipe 160 according to an exemplary embodiment. The construction of the heat pipe 160 and other constructions are identical to those of the above-mentioned exemplary embodiment, and as such a detailed description of different constructions will herein be omitted for convenience of description.

Referring to FIG. 8A, the second contact portion 163 may include many more bent portions 165 than the second contact portion 163 of the above-described exemplary embodiment. One of the second contact portions 163 contacting the second heat radiation plate 140b may include at least four bent portions 165 before reaching the connection portion 164. The other one of the second contact portions 163 contacting the first heat radiation plate 140a is extended from the connection portion 164, and may include at least four bent portions 165.

The higher the number of bent portions 165 contained in the second contact portion 163, the larger the contact region between the second contact portion 163 of the heat pipe 160 and the heat radiation plate 140. Thus, much more heat can be transferred to the heat radiation plate 140, resulting in an increased cooling speed of the ultrasonic probe 1.

The bent portion 165 is not limited to the exemplary embodiment of FIG. 8A, and at least four bent portions 165 may be contained in the second contact portion 163. As the number of bent portions 165 increases, thermal conduction to the heat radiation plate 140 is efficiently achieved. The number of bent portions 165 may be determined in consideration of the external appearance of the housing 10, electronic components provided in the inside of the housing 10, and a space in which a cable (not shown) is provided.

Referring to FIG. 8B, the second contact portion 163 may include a plurality of connection portions 164. One side of the second contact portion 163 contacting the second heat radiation plate 140b may reach the connection portion 164 through several bent portions 165. The other side of the second contact portion 163, which is extended from the connection portion 164 and contacts the first heat radiation plate 140a, is connected to the connection portion 164 facing the second heat radiation plate 140b through several bent portions 165, so that the other side of the second contact portion 163 is re-extended toward the second heat radiation plate 140b. That is, the second contact portion 163 contacts both the first and the second heat radiation plate 140a and 140b via a plurality of connection portions 164.

The bent portions 165 provided at one side and the other side of the second contact portion 163 is not limited to the exemplary embodiment of FIG. 8B, and two or more bent portions 165 may be contained in the second contact portion 163.

Several connection portions 164 may be arranged in parallel at one side as shown in FIG. 8B, in consideration of the external appearance of the housing 10, electronic components provided in the inside of the housing 10, and a space in which a cable (not shown) is provided. The connection portions 164 may be spaced apart from one side or the other side as shown in FIG. 8B. In addition, the number of connection portions 164 may be determined according to the internal structure of the ultrasonic probe 1.

Referring to FIG. 8C, a plurality of heat pipes 160 may be provided instead of a single heat pipe 160 of the above-mentioned exemplary embodiment shown in FIGS. 8A and 8B. When a plurality of heat radiation plates 140 is provided, the number of the heat pipes 160 may be identical to the number of heat radiation plates 140. This exemplary embodiment includes two heat radiation plates (140a, 140b), and the number of heat pipes 160 may be set to two (2).

The heat pipes 160 may include heat generation portions (163a, 163b) respectively contacting the first heat radiation plate 140a and the second heat radiation plate 140b. The heat generation portions (163a, 163b) may include one or more bent portions (165a, 165b) formed to orthogonally bend the extension direction.

Several heat pipes 160 are not limited to the exemplary embodiment of FIG. 8C. If three or more heat radiation plates 140 are provided, the number of heat pipes 160 may be three or more in correspondence with the number of heat radiation plates 140. In addition, the individual heat generation portions (163a, 163b) are not limited to the exemplary embodiment of FIG. 8C, and may include a plurality of bent portions (165a, 165b). The number of the heat pipes 160 and the number of the bent portions (165a, 165b) may be determined in consideration of the external appearance of the housing 10, electronic components provided in the inside of the housing 10, and a space in which a cable (not shown) is provided.

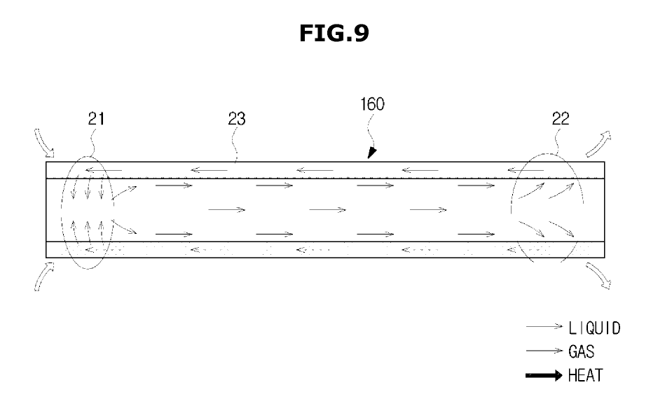

FIG. 9 is a conceptual diagram illustrating the operation principle of heat pipes shown in FIG. 2.

The working fluid is injected into a sealed-pipe-shaped container, and the pipe-shaped container has a vacuum state, resulting in formation of the heat pipe 160.

The working fluid in the heat pipe 160 may have two phases, so that heat can be transmitted through the heat pipe 160.

Referring to FIG. 9, if heat is applied to an evaporator 21 of the heat pipe 160, heat is applied to the inside of the heat pipe 160 by thermal conduction through an outer wall.

The working fluid may be evaporated from the surface of a fine structure (wick) 23 even at a low temperature in a high-pressure heat pipe 160.

The density and pressure of gas are increased in the evaporator 21 due to evaporation of the working fluid, and a pressure gradient is formed in a gas passage of the center part in the direction of a condenser 22 having a relatively low gas density and pressure, so that gas moves along the passage.

In the exemplary embodiment, the moving gas having a large amount of heat corresponding to the evaporation latent heat may move.

The gas flowing in the condenser 22 is condensed at an inner wall of the condenser 22 having a relatively low temperature, so that heat is emitted and returns to a liquid state.

The working fluid having returned to the liquid state may move again toward the evaporator 21 through pores formed in the fine structure 23 by capillary pressure or gravity.

By repetition of the above-mentioned processes, thermal conduction is continuously achieved.

Referring to FIGS. 2 and 6, the ultrasonic probe 1 may further include a probe lower part 170 located at a lower end of the ultrasonic probe 1. Differently from an exemplary embodiment, a heat sink for heat dissipation of the ultrasonic probe 1 may be provided in the probe lower part 170, separately from the heat radiation plate 140. The heat sink may be formed of metal having superior thermal conductivity. The heat radiation plate 140 may be coupled to the heat sink or one side of the heat pipe 160 may be coupled to the heat sink, so that heat generated from the transducer 110 and the driving element 111 can be dissipated.

The ultrasonic probe 1 may further include a cable connection portion 180. The cable connection portion 180 may be coupled to the bottom surface of the body portion 13. A space 181 from which ultrasonic signals are generated may be formed in the cable connection portion 180, and may be coupled to various electronic components (not shown) to obtain measurement values.

As is apparent from the above description, heat generated from an ultrasonic probe is applied to a large region of a heat radiation plate through a heat pipe, so that heat can be effectively radiated to the outside.

In addition, the ultrasonic probe according to the exemplary embodiments can efficiently absorb ultrasonic signals emitted in a backward direction of the ultrasonic probe using micropatterns provided in a heat spreader.

The above-mentioned exemplary embodiments are disclosed only for illustrative purposes. The above-mentioned disclosures are used only to indicate the exemplary embodiments, and the exemplary embodiments can also be used in various combinations, modifications and environments without departing from the scope or spirit of the inventive concept. That is, the exemplary embodiments can be readily modified or changed within the scope of the inventive concept, within the scope equivalent to the disclosed content, and/or within the scope of technology or knowledge well known to those skilled in the art. Therefore, the above-mentioned exemplary embodiments are not intended to limit the scope of the inventive concept.

While exemplary embodiments have been particularly shown and described above, it would be appreciated by those skilled in the art that various changes may be made therein without departing from the principles and spirit of the inventive concept as defined by the following claims.

1. An ultrasonic probe comprising:
a transducer;
a driving element electrically coupled to the transducer;
a backing layer provided underneath the transducer and the driving element in a longitudinal direction of the ultrasonic probe, and configured to absorb heat generated from the transducer and the driving element and to absorb vibrations generated by the transducer;
a heat spreader provided underneath the backing layer in the longitudinal direction of the ultrasonic probe and configured to absorb the heat from the backing layer;
a heat pipe including a first contact portion contacting the heat spreader and a second contact portion in contact with the first contact portion; and
a heat radiation plate configured to contact the second contact portion and transfer the heat from the heat spreader to an exterior of the ultrasonic probe.

2. The ultrasonic probe according to claim 1, wherein the second contact portion comprises a bent portion.

3. The ultrasonic probe according to claim 1, wherein the second contact portion orthogonally extends from the first contact portion, in the longitudinal direction of the ultrasonic probe and pass through the heat radiation plate.

4. The ultrasonic probe according to claim 1, wherein the heat radiation plate includes a first heat radiation plate and a second heat radiation plate arranged facing one another downwardly from the heat spreader along the longitudinal direction of the ultrasonic probe.

5. The ultrasonic probe according to claim 4, wherein the second contact portion is included into a plurality of second contact portions arranged to contact the first and second heat radiation plates and configured to transfer the heat to the first and second heat radiation plates.

6. The ultrasonic probe according to claim 5, wherein the plurality of second contact portions includes a bent portion.

7. The ultrasonic probe according to claim 5, wherein the heat pipe further includes:
a connection portion which extends between the plurality of second contact portions so that one of the plurality of the second contact portions contacting the first heat radiation plate is connected to another one of the plurality of the second contact portions contacting the second heat radiation plate.

8. The ultrasonic probe according to claim 7, wherein:
the connection portion is included into a plurality of connection portions which are arranged in the longitudinal direction of the ultrasonic probe.

9. The ultrasonic probe according to claim 4, wherein the heat pipe is included into a plurality of heat pipes and the heat radiation plate is included into a plurality of heat radiation plates, and
a number of the plurality of heat pipes corresponds to a number of the plurality of heat radiation plates.

10. The ultrasonic probe according to claim 1, wherein:
the first contact portion extends in a direction perpendicular to the longitudinal direction of the ultrasonic probe and is provided in the heat spreader, and
the heat pipe further includes an extension portion which is bent at an end of the first contact portion and extends toward the heat radiation plate.

11. The ultrasonic probe according to claim 10, wherein the extension portion is provided in the heat spreader, and
the heat pipe is bent in the heat spreader and passes through a bottom surface of the heat spreader.

12. The ultrasonic probe according to claim 10, wherein the extension portion is located at an exterior of the heat spreader, and
the heat pipe passes through a side surface of the heat spreader.

13. The ultrasonic probe according to claim 10, wherein the extension portion is included into a plurality of extension portions which are located at opposite sides of the heat spreader and the heat pipe passes through opposite side portions of the heat spreader.

14. The ultrasonic probe according to claim 1, wherein the heat spreader includes a contact portion contacting a bottom surface of the backing layer, and
the contact portion comprises a micropattern having a plurality of holes.

15. The ultrasonic probe according to claim 14, wherein the holes and the contact portion are filled with a thermal grease or a phase change material.

16. The ultrasonic probe according to claim 14, wherein the backing layer has a thickness of about 5 mm or less.

17. The ultrasonic probe according to claim 1, wherein the heat spreader further includes:
a seating portion on which a bottom surface portion and a lateral surface portion of the backing layer are accommodated in the heat spreader.

18. An ultrasonic probe comprising:
a housing configured to house a transducer, a body portion accommodating a driving element configured to drive the transducer, and a handle portion extending from the body portion;
a backing layer arranged underneath the transducer and the driving element in a longitudinal direction of the ultrasonic probe, the backing layer and configured to absorb heat generated by the transducer and the driving element and to absorb vibrations generated by the transducer;
a heat spreader provided underneath the backing layer in the longitudinal direction of the ultrasonic probe and configured to absorb heat from the backing layer;
a heat radiation plate provided inside of the handle portion; and
a heat pipe including
a first contact portion provided inside the heat spreader; and
a second contact portion contacting the heat radiation plate.

19. The ultrasonic probe according to claim 18, wherein the heat radiation plate extends in the longitudinal direction of the ultrasonic probe corresponding to a longitudinal direction of the handle portion.

20. The ultrasonic probe according to claim 18, wherein the heat radiation plate includes a first end provided at a position corresponding to the body portion and a second end located at an opposite side of the first end along a longitudinal direction of the handle portion, and
wherein the second contact portion extends from the first end to the second end, passes through the second end, is bent at the second end, and is extended toward the first end.

21. The ultrasonic probe according to claim 20, wherein the second contact portion further includes a bent portion provided between the first end and the second end.

22. The ultrasonic probe according to claim 18, wherein the heat radiation plate comprises a first heat radiation plate and a second heat radiation plate, respectively corresponding to a first side of the handle portion and a second side opposite to the first side of the handle portion, and
the second contact portion is included into a plurality of second contact portions which respectively contact the first and second heat radiation plates.

23. The ultrasonic probe according to claim 22, wherein the heat pipe further includes:
a connection portion configured to connect one second contact portion of the plurality of second contact portions contacting the first heat radiation plate to another second contact portion of the plurality of second contact portions contacting the second heat radiation plate.

24. The ultrasonic probe according to claim 22, wherein the heat pipe is included into a plurality of heat pipes and the heat radiation plate is included into a plurality of heat radiation plates, and
a number of the plurality of heat pipes corresponds to a number of the plurality of heat radiation plates.

25. The ultrasonic probe according to claim 18, wherein the heat spreader includes a seating portion configured to seat a bottom surface portion and a lateral surface portion of the backing layer, and
a contact surface contacting the bottom surface portion of the backing layer of the seating portion includes a micropattern having a plurality of holes.

26. An ultrasonic probe comprising:
a transducer;
a driving element electrically coupled to the transducer;
a backing layer which is provided underneath the driving element in a longitudinal direction of the ultrasonic probe, contacts a bottom surface portion of the driving element, and is configured to absorb heat generated from the transducer and the driving element and to absorb vibrations generated by the transducer;
a heat spreader which is provided underneath the backing layer in the longitudinal direction of the ultrasonic probe, has a contact portion which contacts a bottom surface portion of the backing layer and is configured to absorb heat from the backing layer;
a heat pipe including a first end provided inside the heat spreader;
a heat radiation plate which contacts the heat pipe; and
micro-sized holes arranged on a surface of the contact portion, which contacts the bottom surface portion of the backing layer.

27. The ultrasonic probe according to claim 26, wherein the micro-sized holes and the contact portion are filled with a thermal grease or a phase change material.
